(19) United States
(12) Patent Application Publication
(10) Pub. No.: US 2017/0023542 A1
(43) Pub. Date: Jan. 26, 2017

(54) Smartphone dock and diagnostic-test reader plus related methods

(71) Applicants: Naishu Wang, San Diego, CA (US); Jimmy Jing, San Diego, CA (US)

(72) Inventors: Naishu Wang, San Diego, CA (US); Jimmy Jing, San Diego, CA (US)

(21) Appl. No.: 14/809,262

(22) Filed: Jul. 26, 2015

(51) Int. Cl. G01N 33/487; A61B 5/15; G06K 9/00; G01N 21/78; H04N 5/225; A61B 10/00; H04M 1/04

(52) U.S. Cl. CPC G01N 33/487; A61B 10/0051; A61B 5/15; A61B 10/007; A61B 10/0038; A61B 10/0064; H04M 1/04; G01N 21/78; H04N 5/2257; G06K 9/00671; A61B 2010/0074

(57) ABSTRACT

Disclosed is are a diagnostic test reader and related methods or systems. In one embodiment, a smartphone, including diagnostic software and a camera, may be docked onto the reader so that the camera of the phone is coupled to a diffuser for imaging test strips or other test cartridges provided to the reader. Preferably, the diagnostic software is configured to interpret an image of the strip and provide diagnostic feedback via the interface of the phone. Light for producing the image may be from the flash of the smartphone camera or a light source inside the reader. The more specific details of the disclosed reader are described in connection with the figures.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

Field of Invention

The disclosed subject matter is in the field of smartphone based diagnostic-test readers, and related methods.

Background of the Invention

Diagnosis and monitoring of biochemical or physiological conditions (including the general health) of humans and plant life or wildlife can involve the sampling and testing of biological samples (e.g., saliva, blood, urine, fecal matter, foodstuffs, minerals, plantstuffs, etc.). Diagnostic test strips, including lateral flow chromatographic immunoassay devices, are useful on-site for quickly obtaining a variety of qualitative results relating to biochemical and physiological conditions of things without laboratory equipment. However, diagnostic test strips, by themselves, only provide visual cues to whether or not a test substance is present in a sample above a given detection limit. In other words, human errors may occur during result interpretation by the naked eye.

In view of the foregoing limitations of visually observing test strip results and the ubiquity of smartphones in modern societies, test readers and related software applications have been devised that incorporate the imaging (camera) and computing hardware of a smart phone to interpret the results of a test strip. Typically, such a reader features: (1) a test strip holder that is positioned over the camera and light source (or other sensors) of the smartphone for imaging of the test strip result, and (2) software installed on the computer hardware of the smartphone which interprets test results from images of the test strip. The methodologies of using these devices allow for more accurate or specific readings and interpretations of test strip results and have generally advanced the state of the art of on-site testing and diagnostics of biological samples.

While the methodologies associated with known smartphone test readers allow for improved interpretation of test strip results, the structural features of known test readers have not been satisfactory in all situations. Specifically, the structures of known test readers can promote cross-contamination between the test strip and either the smartphone or the user's hand because the strip must be inserted into the reader while the smart phone is in hand and the smart phone must be subsequently manipulated to run the test interpretation and software applications. This is particularly worrisome for either sensitive tests or repulsive test samples (like fecal matter or blood).

One solution for avoiding cross-contamination between the phone and test strip is encasement of the smartphone by the reader. However, encasing the smartphone can be tedious and time consuming so that either (i) a test cannot be quickly performed and assessed or (ii) the smartphone is inoperable for purposes other than test strip reading. Furthermore, encasing the smartphone does not eliminate the possibility of cross-contamination between the user's hand and the test strip. Thus, a need exists for smartphone test readers which minimize hand interaction for operation and which do not otherwise promote cross-contamination between the smartphone or user's hand and test strip.

The structures of known smartphone test readers are also problematic in view of the varying sizes and shapes of smartphones. Known smartphones are either tailored to a specific smartphone type or require complicated or nuanced smartphone mounting mechanisms. Therefore, a need further exists for smartphone test readers that are universal to all types of smart phones, without the need for complicated or nuanced attachment mechanisms.

Known smartphone readers are additionally limited by structure to a specific test result interpretation methodology. For instance, some methodologies for interpreting test strip results require fluorescent lighting, but smartphones lack a fluorescent light source whereby test readers cannot interpret results for such methodologies unless there is a fluorescent light source built-in to the reader. As a consequence, a need also exists for smartphone test readers that can be adapted to multiple test interpretation methodologies.

Finally, known test readers and related methods can only interpret test results. Without more functionality than test result interpretation, test readers cannot speed-up or otherwise enhance the test strip results. A test reader with related methods that could encourage or facilitate faster test results is desirable.

### SUMMARY OF THE INVENTION

In view of the foregoing, it is an object of this disclosure to describe a smartphone test reader that allows limited hand interaction for operation and which does not otherwise promote cross-contamination between the smartphone or user's hand and test strip. It is a further objective of this disclosure to describe smartphone test readers that are universal to a variety of smart phone types without the need for overly complicated or nuanced attachment mechanisms. Yet still, an objective of this description is to disclose smartphone test readers that can be adapted to multiple test interpretation methodologies. Finally, it is an object of this disclosure to describe smart phone test readers that are capable of encouraging or facilitating faster test results.

Disclosed is a diagnostic test reader and related methods or systems. In a preferred embodiment, a smartphone, including diagnostic software and a camera, may be docked onto the reader so that the camera of the phone is coupled to a diffuser for imaging test strips or other test cartridges provided to the reader. In one embodiment, the software installed on phone can interpret an image of the test strip and provide diagnostic feedback via the interface of the phone. For instance, the software may examine images of the test strip or cartridge for visual and chemical markers. Light for producing the image may be from the flash of the smartphone camera or a light source inside the reader (e.g., fluorescent lamps). In one embodiment, a light source is triggered by docking of the smartphone. In a preferred embodiment, the docking station of the reader may be customized to a particular smartphone or universal to all smartphones with a camera. A docking station for a smartphone on a reader is preferable to a mere attachment of the reader to a smartphone because the cell phone is less susceptible to contaminants from the test strips and vice-versa. In one embodiment, the docking station of the disclosed reader allows for use of additional light sources beyond the flash-light source of the smartphone. Furthermore, when docked in a reader, the smart phone vibration function can be used, for example, to mix fluids or agitate the test strips. Test strips may be inserted into the reader below the camera of a docked smartphone.

It is yet another object of the present application to meet the aforementioned needs without any of the drawbacks associated with apparatus heretofore known for the same purpose. It is yet still a further objective to meet these needs in an efficient and inexpensive manner.

### BRIEF DESCRIPTION OF THE FIGURES

The manner in which these objectives and other desirable characteristics can be obtained is better explained in the following description and attached figures in which:

FIG. 1 is a perspective view of a smartphone test reader 1000;

FIG. 2 is a front view of the smartphone test reader 1000 of FIG. 1;

FIG. 3 is a back view of the smartphone test reader of FIG. 1;

FIG. 4 is a .right-side view of the smartphone test reader of FIG. 1;

FIG. 5 is a left-side view of the smartphone test reader of FIG. 1

FIG. 6 is a top view of the smartphone test reader 1000 of FIG. 1;

FIG. 7 is a bottom view of the smartphone test reader 1000 of FIG. 1;

FIG. 8 is an environmental view of the smartphone test reader 1000 of FIG. 1;

FIG. 9 is an exploded view of the smartphone test reader 1000 of FIG. 1;

FIG. 9A is a cross section of the smart phone test reader of FIG. 1;

FIG. 9B is a cross section of the smart phone test reader of FIG. 1;

FIG. 10. Is an assembly of a smart phone 2000 and the smartphone test reader of FIG. 1;

FIG. 11 is a horizontal cross section of the assembly of FIG. 10;

FIG. 12 is a longitudinal cross section of the assembly of FIG. 10;

FIG. 13 is a screen shot of software for interpreting test results on a smartphone;

FIG. 14 is a screen shot of software for interpreting test results on a smartphone;

FIG. 15 is a screen shot of software for interpreting test results on a smartphone;

FIG. 16 is a screen shot of software for interpreting test results on a smartphone;

FIG. 17 is a screen shot of software for interpreting test results on a smartphone;

FIG. 18 is a screen shot of software for interpreting test results on a smartphone;

FIG. 19 is a screen shot of software for interpreting test results on a smartphone;

FIG. 20 is a screen shot of software for interpreting test results on a smartphone;

FIG. 21 is a screen shot of software for interpreting test results on a smartphone;

FIG. 22 is a screen shot of software for interpreting test results on a smartphone;

FIG. 23 is a screen shot of software for interpreting test results on a smartphone;

FIG. 24 is a screen shot of software for interpreting test results on a smartphone;

FIG. 25 is a screen shot of software for interpreting test results on a smartphone;

FIG. 26 is a screen shot of software for interpreting test results on a smartphone;

FIG. 27 is a screen shot of software for interpreting test results on a smartphone; and

FIG. 28 is a screen shot of software for interpreting test results on a smartphone.

It is to be noted, however, that the appended figures illustrate only typical embodiments disclosed in this application, and therefore, are not to be considered limiting of its scope, for the invention may admit to other equally effective embodiments that will be appreciated by those reasonably skilled in the relevant arts. Also, figures are not necessarily made to scale.

### DETAILED DESCRIPTION OF PREFERRED EMBODIMENTS

Disclosed is a diagnostic test reader and related methods or systems. In one embodiment, a smartphone, including diagnostic software and a camera, may be docked onto the reader so that the camera of the phone is coupled to a diffuser for imaging test strips or other test cartridges provided to the reader. Preferably, the diagnostic software is configured to interpret an image of the strip and provide diagnostic feedback via the interface of the phone. Light for producing the image may be from the flash of the smartphone camera or a light source inside the reader. The more specific details of the disclosed reader are described in connection with the figures.

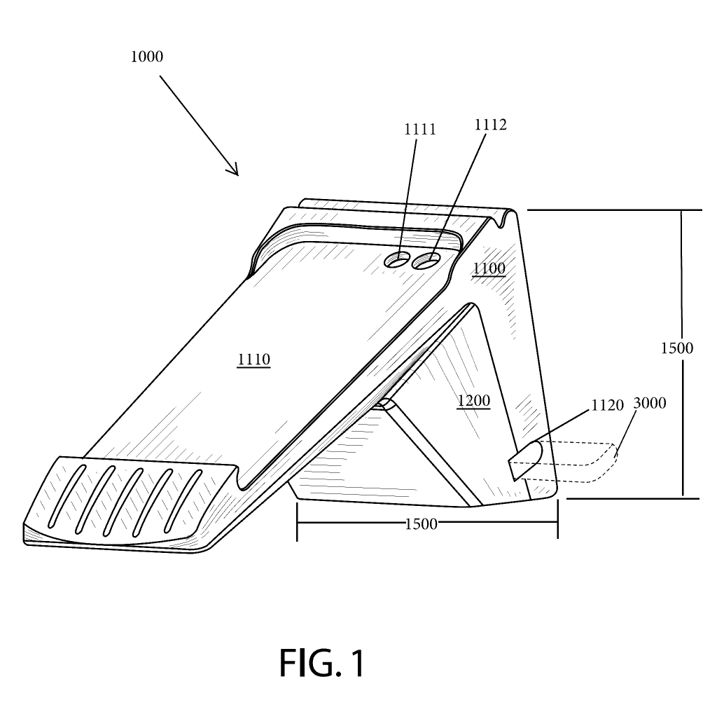

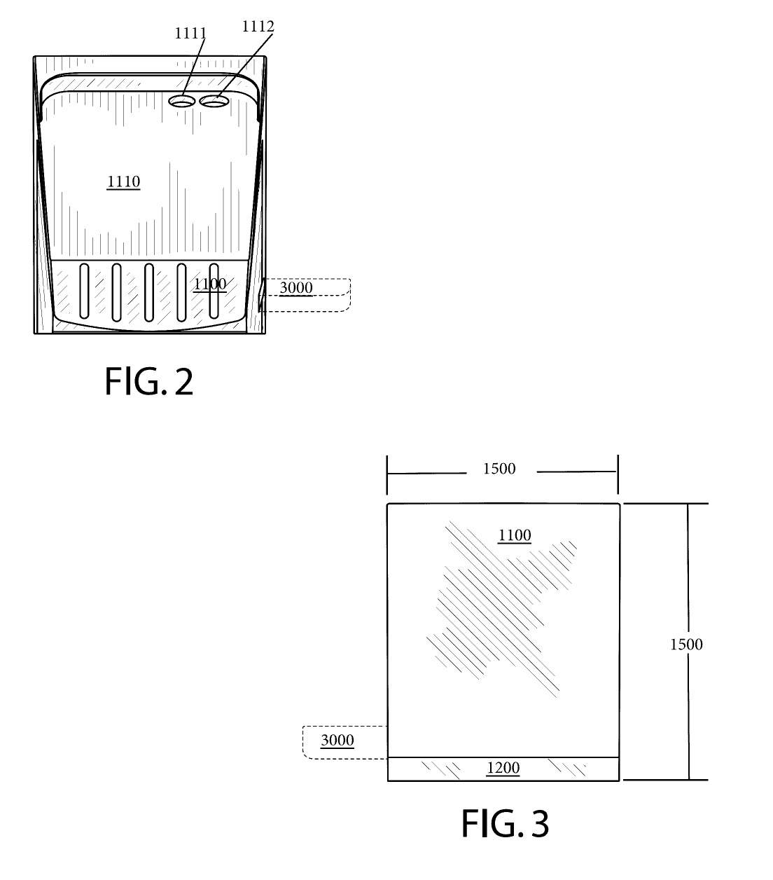

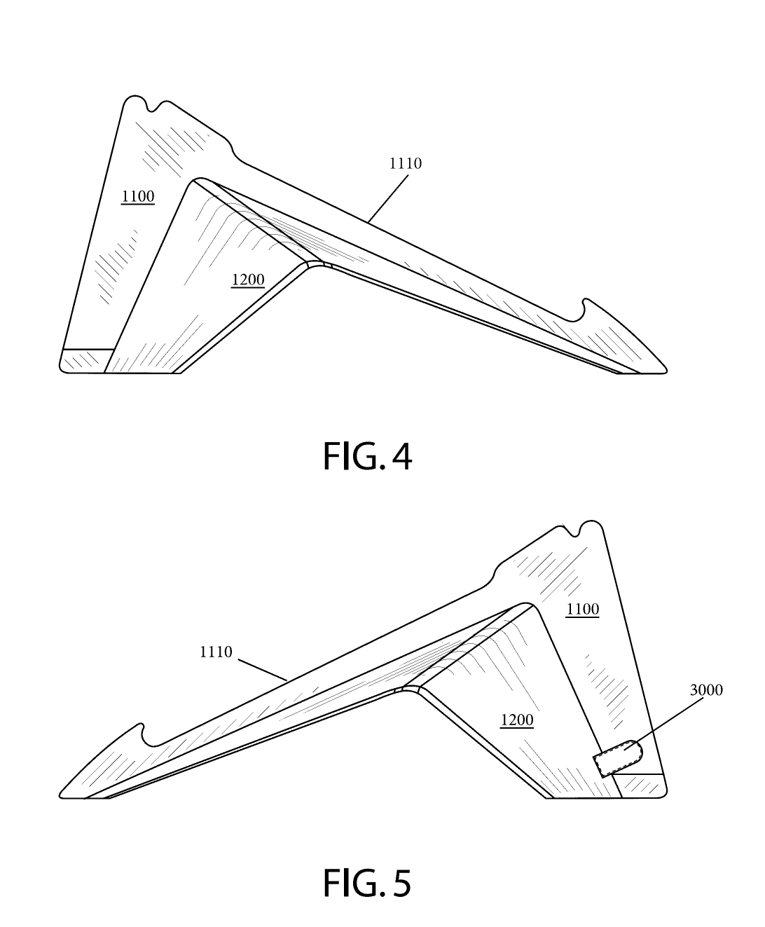

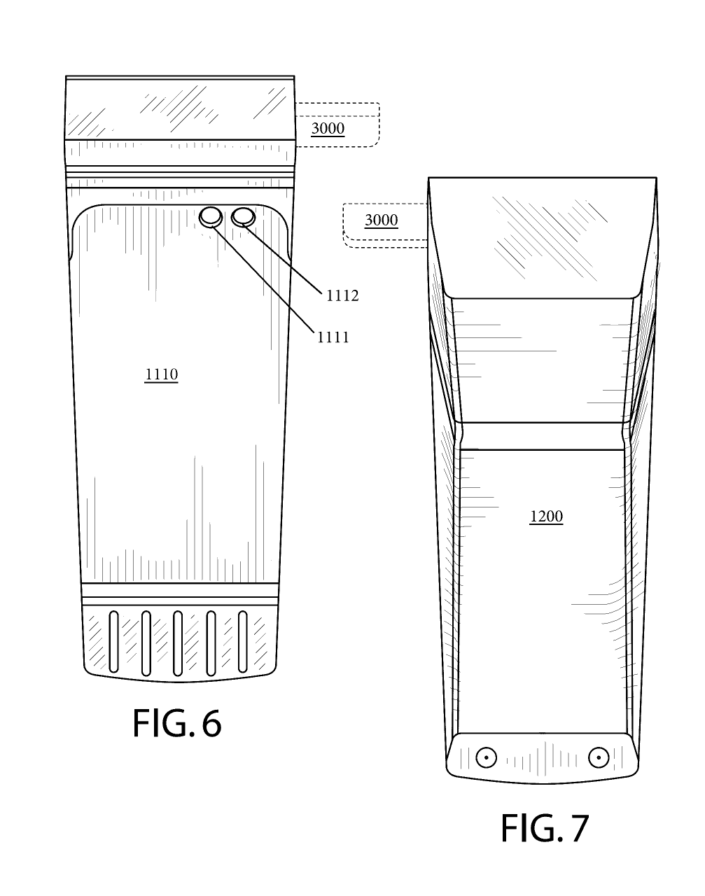

FIG. 1 is a perspective view of a smartphone test reader 1000. FIGS. 2 through 7 are respectively front, back, right-side, left-side, top, and bottom views of the smart phone test reader 1000 of FIG. 1. Together, these figures illustrate the preferred external structures of the depicted smartphone test reader 1000. As shown, the reader 1000 is generally in the form of an easel that is defined by two components: a top panel 1100 and a bottom panel 1200. The top 1100 and bottom 1200 panel mate to form an upright in the back (see FIG. 3) and an angled support surface or docking station 1110 in the front (see FIG. 2) for a smartphone (not shown). It should be noted that hereinafter the terms “upright” or “upright rear portion” designate the back portion 1500 of the reader 1000, shown in FIGS. 1 and 3, i.e., the portion 1500 that supports the docking station 1110 in an angled orientation. The upright rear portion of the reader 1000 is configured to receive a test cartridge 3000 or test strip via a port 1120. As shown, the docking station has a diffuser aperture 1111 and a lens aperture 1112.

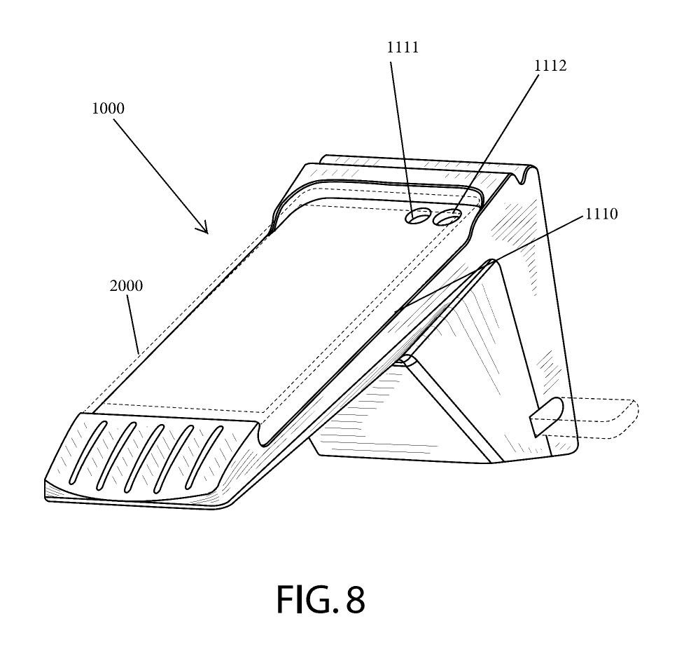

FIG. 8 is an environmental view of the reader 1000. As discussed in greater detail below, the docking station 1110 is configured to receive a smartphone 2000 with a light source and camera lens that that are mated or coupled with the diffuser aperture 1111 and lens aperture 1112.

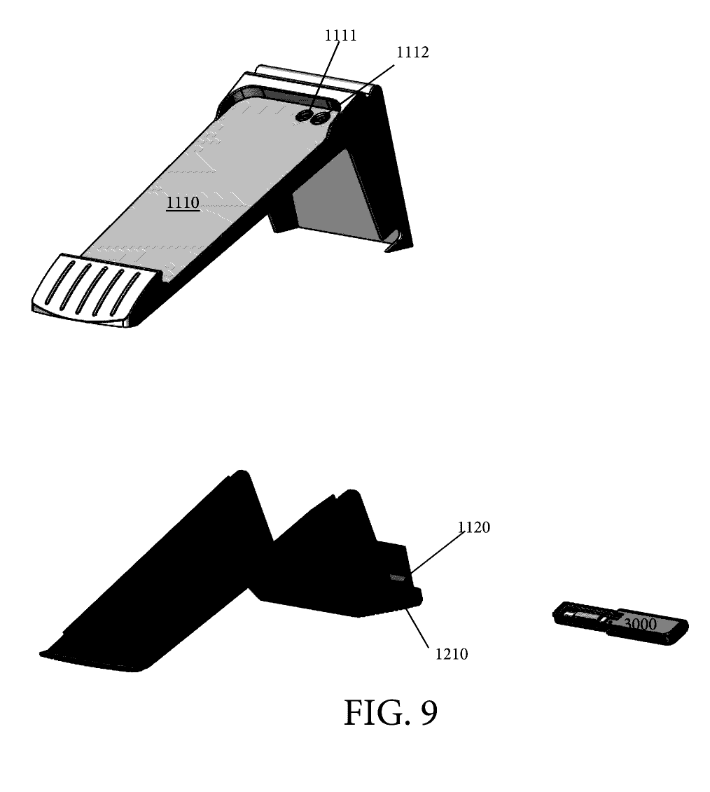

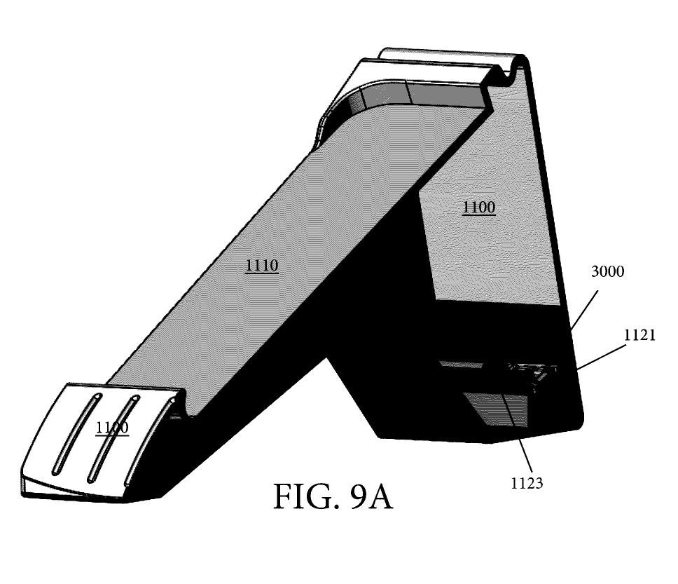

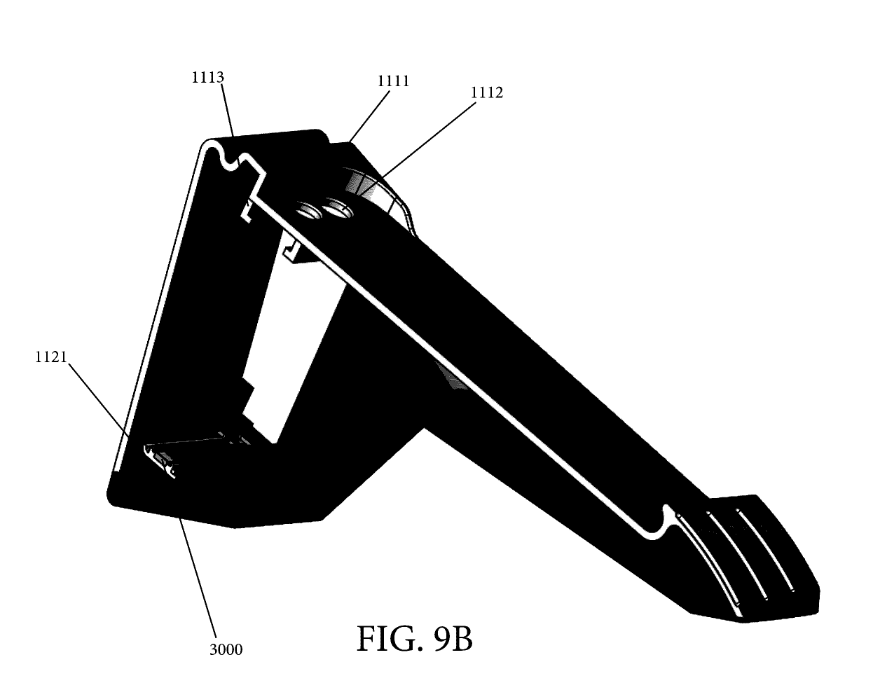

FIG. 9 is an exploded view of the reader 1000 of FIGS. 1 through 8. FIGS. 9A and 9B are left and right hemispheres of the reader 1000 with the inside of the reader exposed. The figures illustrate the mating of the upper and lower panels 1100, 1200 and the internal structural features of the reader 1000. As shown in FIG. 9, the upper panel 1100 receives the bottom panel (see FIGS. 9A and 9B) and accomplishes a snap fit or friction fit. As shown in FIGS. 9, 9A, and 9B, the lower panel 1200 features a deck or platform 1121 that is aligned with the external port 1120 for receiving, supporting and presenting a cartridge 3000 to the diffuser and lense ports 1111, 1112 of the upper panel 1100. Referring to FIG. 9A, the deck 1121 features a sensor switch 1123 that, as discussed further below, wirelessly alerts a smartphone (not shown) placed in the docking station 1110 that a cartridge 3000 has been placed in the deck 1121. Referring to FIG. 9B, the upper panel 1100 features a diffuser holder 1113 for holding a diffuser (not shown) between a cartridge 3000 and a light source of a smartphone (not shown) installed in the docking station 1110.

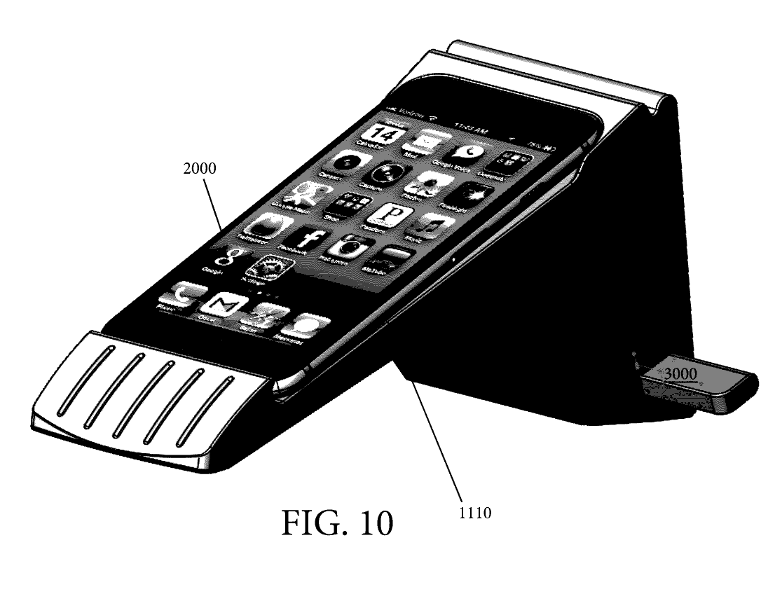

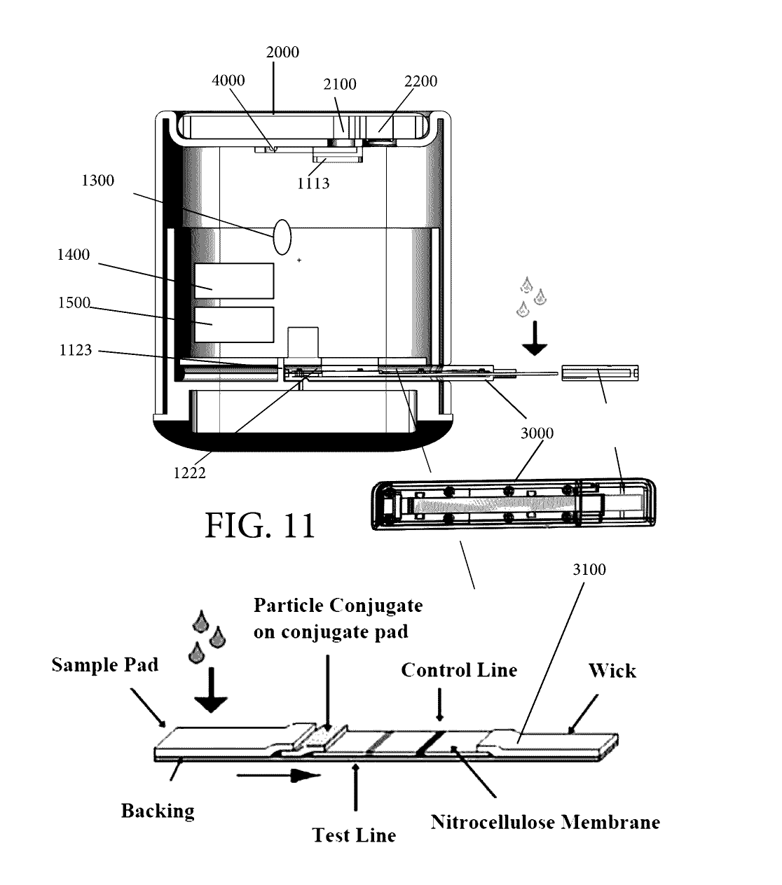

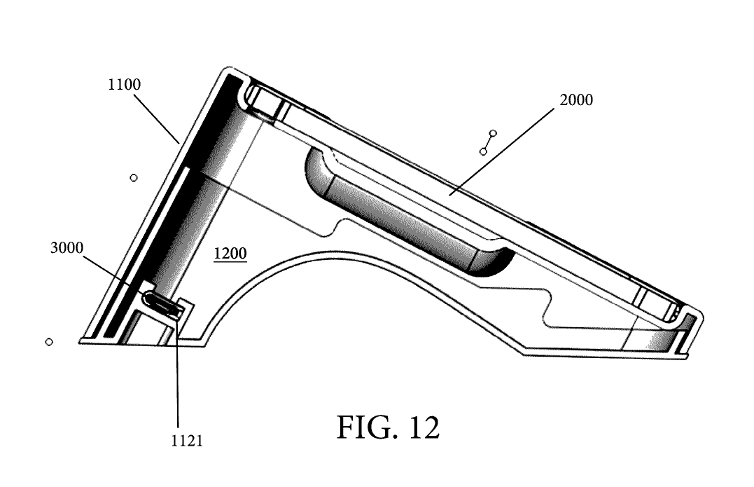

FIG. 10 is a perspective view of an assembly of a unified smartphone 2000 and a smartphone test reader 1000. FIG. 11 is a horizontal cross section of the assembly of FIG. 10. FIG. 12 is a longitudinal cross section of the assembly of FIG. 10. As shown in FIG. 10, (a) a smartphone 2000 may be positioned in the docking station 1110 of the reader's 1000 upper panel 1100 and (b) a cartridge may be provided to the port 1120 in the upright back end of the reader 1000.

As shown in FIGS. 11 and 12, a smart phone 2000 may be positioned in the docking station 1100 so that the flash 2100 of the smartphone 2000 is coupled to the diffuser aperture 1111 and the smartphone's 2000 camera lens 2200 is coupled to the lens aperture 1112. Suitably, the diffuser holder 1113 may be provided with a diffuser 4000. In one embodiment, the test cartridge 3000 is provided through the port 1120 so that the cartridge 3000 rests in the deck 1121 and triggers the switch 1123 for alerting the smartphone of its presence. In one embodiment, (a) an additional light source (e.g., a fluorescent bulb) 1300, (b) an additional detection system (e.g., a camera) 1400, and (c) a power source (e.g. rechargeable battery) 1500 are provided to within the internal cavity of the reader 1000. Suitably, the deck 1121 presents the cartridge 3000 to the cameras 1500 or 2200 and light sources 1400 or 2100 for imaging.

FIG. 11 through 12 further illustrate a typical mode of operation for the reader 1000. Initially a smart phone 2000 may be provided to the docking station 1110 and a cartridge 3000 provided to the port 1200. In a preferred embodiment, the reader 1000 features wireless communication and sensor hardware (not shown) that senses the docking of the phone 2000 and prompts initialization of the light source 2100 of the smartphone 2000 or the internal light source 1400. A sample (shown as drops) may be provided to the test strip 3000 as shown. In one embodiment, the reader 1000 senses the presence of the cartridge 3000 via the sensor switch 1123 and wirelessly commands the phone 2000 to initiate its vibrating function so that the reaction or other processes associated with testing the sample can be facilitated by the motions of the vibrating smartphone. In lateral flow test strips, the vibration function of the smartphone may be used to facilitate reactions or otherwise make the test strip more sensitive. Finally, the interface of the smartphone 2000 may be interacted with to assess the test results. In a preferred embodiment, the software and smartphone may feature audible commands for walking a user through the test reading procedures.

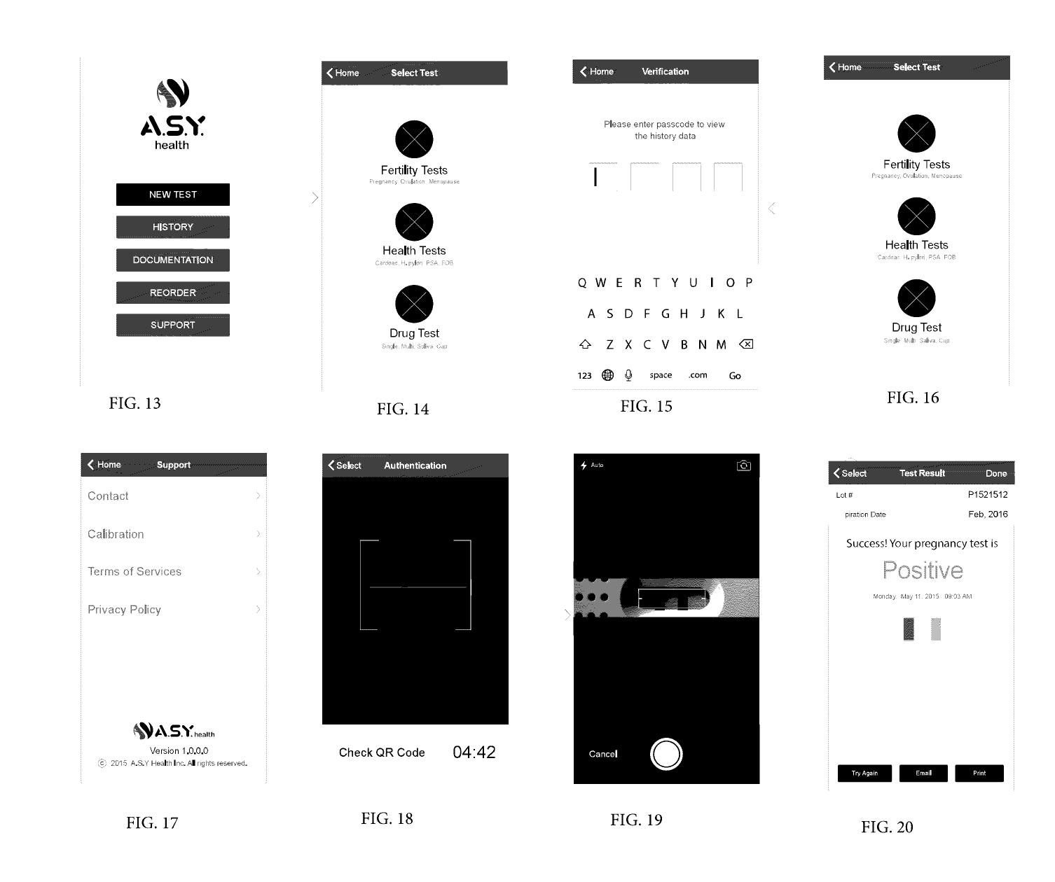

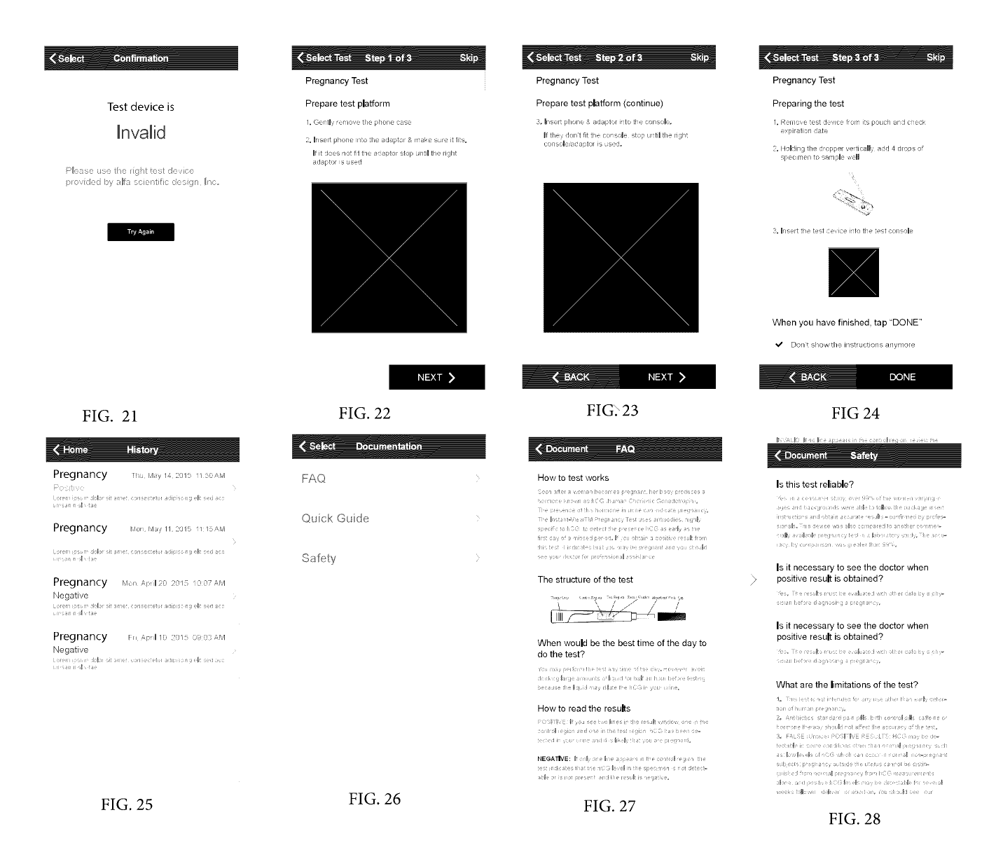

FIGS. 13 through 28 illustrate various screenshots of a software applications that may be installed on a smartphone for reading test results. FIG. 13 illustrates a home screen for the software app. As shown, the home screen features at least three buttons: a new test button; a history button; and a support button. Interacting with the new test button will result in the Select Test screenshot shown in FIG. 14. Referring to that figure, a list of possible tests are presented for having the results read by the reader 1000 of the earlier figures. In a preferred embodiment, a Fertility Tests option may be selected so that a user is given a choice to either (a) scan a QR code or a test to see if the test strip or cartridge is authorized for use in the reader or (b) get a guide for administering the test. IF scanning a QR code is selected, then the software transitions to the Authentication screen shot of FIG. 18. IF the get guide options are selected, THEN the screen shots of FIGS. 22 through 24 (guides for administering the test) are presented in series AND the software transitions to the Authentication screen shot of FIG. 18. IF the test strip or cartridge is authenticated by the software and QR scan function of the smartphone, T the software presents the imaging screen shot of FIG. 19, ELSE an invalid screenshot FIG. 21 is presented. Referring to the screenshot of FIG. 19, images of the test cartridge 3000 of the earlier figures may be captured, analyzed and presented in the results screenshot (FIG. 20). In the case of a fertility test, the image is analyzed for two bars (positive), a left bar (negative), or a right or no bars (inconclusive).

Returning to the home screen of FIG. 13, IF the history command button is selected, THEN a password entry screen (FIG. 15) is presented to the user wherein IF a password is successfully entered, THEN a history of tests is presented (FIG. 25). Still referring to FIG. 13, if the documentation command is selected, THEN a user is presented with options (e.g FIG. 14) to view the documentation of one of the various tests. For example, if a fertility test is selected, a user can choose, via the screenshot of FIG. 26, to view FAQ (FIG. 27), safety information (FIG. 28) or a guide (FIGS. 22 through 24). In one embodiment, the software further features functionality for calendaring the results of the test and, in the case of fertility, periods of time where the user is fertile.

While the invention has been shown in a preferred embodiment, including a generally easel-type test reader, it is recognized that departures may be made from the subject matter as described while nevertheless remaining within the spirit of this invention. The invention is therefore not to be limited except as set forth in the claims which follow.

Although the method and apparatus are described above in terms of various exemplary embodiments and implementations, it should be understood that the various features, aspects and functionality described in one or more of the individual embodiments are not limited in their applicability to the particular embodiment with which they are described, but instead might be applied, alone or in various combinations, to one or more of the other embodiments of the disclosed method and apparatus, whether or not such embodiments are described and whether or not such features are presented as being a part of a described embodiment. Thus the breadth and scope of the claimed invention should not be limited by any of the above-described embodiments.

Terms and phrases used in this document, and variations thereof, unless otherwise expressly stated, should be construed as open-ended as opposed to limiting. As examples of the foregoing: the term “including” should be read as meaning “including, without limitation” or the like, the term “example” is used to provide exemplary instances of the item in discussion, not an exhaustive or limiting list thereof, the terms “a” or “an” should be read as meaning “at least one,” “one or more,” or the like, and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that might be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases might be absent. The use of the term “assembly” does not imply that the components or functionality described or claimed as part of the module are all configured in a common package. Indeed, any or all of the various components of a module, whether control logic or other components, might be combined in a single package or separately maintained and might further be distributed across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives might be implemented without confinement to the illustrated examples. For example, block diagrams and their accompanying description should not be construed as mandating a particular architecture or configuration.

All original claims submitted with this specification are incorporated by reference in their entirety as if fully set forth herein.

We claim:

1. A smartphone test reader comprising:
an upright;
a docking station;
wherein the upright is configured to receive a test cartridge or test strip via a port;
wherein the docking station has a diffuser aperture and a lens aperture; and
wherein the docking station is configured to receive a smartphone with a light source and a camera lens so that that the light source and camera lens are respectively mated or coupled with the diffuser aperture and lens aperture.

2. The reader of claim 1 further comprising:
wherein the upright internally features a deck or platform that is aligned with the port for receiving, supporting and presenting a cartridge to the diffuser and lense apertures.

3. The reader of claim 2 further comprising:
a sensor switch that is configured to detect a cartridge provided to the deck and alerts a smartphone placed in the docking station that the cartridge has been placed in the deck.

4. The reader of claim 3 further wherein the switch is configured to trigger a vibrating function of a smartphone.

5. The reader of claim 3 further comprising a diffuser holder for holding a diffuser between a cartridge positioned in the deck and the light source of a smartphone installed in the docking station.

6. A method of testing comprising the steps of:
positioning a smartphone in a docking station of a test reader so that a flash light source of the smartphone is coupled to a diffuser aperture of the test reader and so that a camera lens of the smartphone is coupled to a lens aperture of the test reader;
providing a diffuser holder of the test reader with a diffuser so that the diffuser is positioned adjacent to said diffuser aperture and operates to diffuse the light source of the camera;
providing a test cartridge through a port in an upright of the test reader so that the cartridge rests in a deck and triggers a sensor for alerting the smartphone of the presence of the cartridge;
wherein the deck presents the cartridge to the camera and light source for imaging;
capturing an image of the cartridge via the camera and storing the image on computer memory of the smartphone;
analysing the image via software to determine a test result; and,
presenting the test result on a display of the smartphone.

7. The method of testing in claim 6, wherein the testing and image relate to testing of any of the group of body fluids consisting essentially of either: saliva, blood, urine, fecal matter, sweat, or vaginal.

8. The method of testing of claim 6 wherein the software for analyzing the image is stored on the smartphone in an application.
